(19) United States
(12) Patent Application Publication
(10) Pub. No.: US 2006/0073103 A1
(43) Pub. Date: Apr. 6, 2006

(54) Controlled Dosage Aerosols With Lecithin As Surface-Active Agent

(76) Inventors: Franz Guck, Rheinfelden (DE); Gieselher Warnke, Herrischried (DE)

Correspondence Address: D Peter Hochberg, 1940 East 6th Street, 6th Floor, Cleveland, OH, 44114, US

(21) Appl. No.: 10/540,648

(22) PCT Filed: Dec. 24, 2003
(86) PCT No.: PCT/EP03/14901

§ 371 Date: Jun. 24, 2005

(30) Foreign Application Priority Data
Dec. 24, 2002 (DE) 102 60 882.2

(51) Int. Cl. A61L 9/04; A61K 31/573

(52) U.S. Cl. 424/45; 514/179

(57) ABSTRACT

A controlled dosage aerosol containing at least one medicinal agent, pressure-liquefied isobutane as the propellant, and lecithin as the surfactant.

### CROSS-REFERENCE TO RELATED APPLICATIONS

This application is a National Stage application of International Application No. PCT/EP2003/014901, filed on Dec. 24, 2003, which claims priority of German application number 102 60 882.2, filed on Dec. 24, 2002.

### BACKGROUND OF THE INVENTION

1. Field of the Invention

The subject matter of the present invention is controlled dosage aerosols which comprise at least one medicinal agent as well as a mixture made of pressure-liquefied isobutane as propellant and lecithin as surface-active agent.

The present invention more particularly relates to controlled dosage aerosols comprising at least one medicinal agent with anti-asthmatic action from the group of the glucocorticoids as well as a mixture made of pressure-liquefied isobutane as propellant and lecithin.

2. Description of the Prior Art

Aerosol pressurized gas packs, also called controlled dosage aerosols or metered aerosols for short, which are produced and employed by using liquefied pressure gases or compressed gases as a propellant, have been known for a long time. Generally, such metered aerosols consist of a pressure vessel, such as of metal or glass, having a valve construction for withdrawing its content, and of the actual agent to be sprayed, which in most cases consists of an active substance solution as well as a propellant in the form of a gas or a gas mixture liquefied under pressure. The pressure-liquefied gas or pressure-liquefied gas mixtures should ideally be miscible with the active substance at any ratio so that one single liquid phase is formed. As an alternative, the pressure-liquefied gas or gas mixture should form a suspension with the active substance which can be shaken easily and above which a gas phase forms. Depending on the agent contained, these metered aerosols are used in the cosmetic and medical fields, or as a room spray, insecticidal spray, and the like.

The propellants of controlled dosage aerosols have to satisfy special requirements. They must by no means react with the components of the active agent solution. Also, the propellants must be non-irritating and non-toxic. Fluorochlorinated hydrocarbons had proved particularly suitable. However, because of their ozone-depleting effect it has been necessary to develop alternative propellants.

However, the quality of these alternative agents must be comparable to that of the fluorochlorinated hydrocarbons; above all, they must be both non-injurious to health and ecologically compatible. Initially, partially halogenated fluorochlorinated hydrocarbons were frequently propagated as replacements but these still have an unacceptably high ozone-depleting ability.

DE 41 32 176 discloses controlled dosage aerosols for administering isoprenalin derivatives, the so-called β-sympathomimetics, or the non-steroid anti-inflammatory agent DNCG wherein isobutane is employed as a propellant.

DE 199 11 064 discloses controlled dosage aerosols containing broncholytic and/or anti-inflammatory agents from the group of the glucocorticoids, with isobutane as propellant and oleic acid or Span 85 as surface-active substances. These dosage aerosols, however, have the disadvantage of a dissatisfactory resuspensibility and of too quick a sedimentation of the active substance in the propellant.

### SUMMARY OF THE INVENTION

An object of the present invention to provide a controlled dosage aerosol for medicinal agents, especially for anti-asthmatic medicinal agents from the group of the glucocorticoids which does not have the disadvantages of the controlled dosage aerosols known from DE 199 11 064.

### DETAILED DESCRIPTION OF THE INVENTION

It was surprisingly found that the adjuvant lecithin leads to a marked improvement of the resuspensibility of medicinal agents, especially of glucocorticoids in isobutane.

Lecithins are glycerophospholipides that are made from fatty acids, glycerol, phosphoric acid and choline. Naturally occurring lecithins are derivatives of 1,2-diacyl-sn-glycerol-3-phosphoric acid. When extracting lecithin from biological material one always obtains a mixture of lecithins that differ from each other by the different fatty acid esters.

According to the present invention, an advantageous lecithin is soybean lecithin, which is already widely used in the pharmaceutics industry as an emulsifier.

In a comparison of the sedimentation behaviour of suspensions of medicinal agent in isobutane under addition of soybean lecithin or various surface-active agents commonly employed in the production of anti-asthmatic metered aerosols it was observed, as will be seen from the below example, that the medicinal agent suspension with soybean lecithin took 10 times longer to sediment than a medicinal agent suspension with oleic acid, and 5 times longer than a medicinal agent suspension with Span 85.

In further tests, with a ratio of medicinal agent to soybean lecithin of 1:2, 1:1 or 1:0.5, no differences were observed in the sedimentation time, so that a ratio of medicinal agent to soybean lecithin of 1:0.5 may advantageously be chosen.

### EXAMPLE

Comparison of the suspension behaviour of suspensions of medicinal agent in isobutane, using various surface-active agents

| | Relative |
|---|---|
| | sedimentation |
| Glucocorticoid:oleic acid (100:1) | 1 |
| Glucocorticoid:Span 85 (1:1) | 2 |
| Glucocorticoid:soybean lecithin (1:2) | 10 |
| Glucocorticoid:soybean lecithin (1:1) | 10 |
| Glucocorticoid:soybean lecithin (1:0.5) | 10 |

In further tests, the following formulations have turned out to be especially advantageous:

| | | |
|---|---|---|
| Formulation 1: | Glucocorticoid | 0.1%-0.2% |
| | Lecithin | 0.05%-0.4% |
| | Isobutane | 99.85%-99.4% |
| Formulation 2: | Glucocorticoid | 0.5%-1.0% |
| | Lecithin | 0.25%-4.0% |
| | Isobutane | 99.75%-95.0% |

For the glucocorticoid beclomethasone diproprionate, the following formulation has been found useful:

| | | |
|---|---|---|
| Formulation 3: | Beclomethasone | 0.1%-2.5% |
| | Soybean lecithin | 0.05%-5.0% |
| | Isobutane | 99.85%-92.5% |

For budesonide the following formulation has been found extremely useful:

| | | |
|---|---|---|
| Formulation 4: | Budesonide | 0.1%-2.5% |
| | Soybean lecithin | 0.05%-5.0% |
| | Isobutane | 99.85%-92.5% |

All quantities relate to percent by weight.

The inventive aerosols may be prepared by mixing the various components under conditions in which the propellant and the surfactant are liquid and in which the active agent is present in a solid phase.

The suspension of medicinal agent is filled through the valve into the clinched tin under pressure, which tin at the beginning of the filling process has room temperature. The suspension has a temperature of approx. −10 to +10° C. Subsequently, the tin is filled up with the propellant, thereby cleaning the valve at the same time.

The controlled dosage aerosols according to the present invention may be used in the treatment of humans and animals, in particular in the treatment of allergic diseases of the respiratory tract, such as asthma or allergic rhinitis (hay fever), such as by oral or nasal inhalation.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A controlled dosage aerosol comprising at least one medicinal agent, a propellant, and a surface-active agent, said surface-active agent being lecithin, wherein said at least one medicinal agent is in the form of a suspension and said propellant is pressure-liquefied isobutane.

2. The controlled dosage aerosol according to claim 1, wherein said at least one medicinal agent is at least one glucocorticoid selected from the group consisting of cortisol, prednisone, prednisolone, methylprednisolone, triamcinolone, prednylidene, fluocortolone, paramethasone, dexamethasone, betamethasone, flunisolide, fluticasone, beclomethasone, budesonide and anti-asthmatically active derivatives thereof.

3. The controlled dosage aerosol according to claim 1, wherein said controlled dosage aerosol comprises glucocorticoid in the amount of 0.1%-0.2%, lecithin in the amount of 0.05%-0.4% and isobutane in the amount of 99.85%-99.4%.

4. The controlled dosage aerosol according to claim 1, wherein said controlled dosage aerosol comprises glucocorticoid in the amount of 0.5%-1.0%, lecithin in the amount of 0.25%-4.0% and isobutane in the amount of 99.75%-95.0%.

5. The controlled dosage aerosol according to claim 1, wherein said lecithin is soybean lecithin.

6. The controlled dosage aerosol according to claim 1, wherein said controlled dosage aerosol comprises beclomethasone in the amount of 0.1%-2.5.0%, soybean lecithin in the amount of 0.05%-5.0% and isobutane in the amount of 99.85%-92.5%.

7. The controlled dosage aerosol according to claim 1, wherein said controlled dosage aerosol comprises budesonide in the amount of 0.1%-2.5.0%, soybean lecithin in the amount of 0.05%-5.0% and isobutane in the amount of 99.85%-92.5%.

8. The controlled dosage aerosol according to claim 5, wherein the ratio of glucocorticoid and soybean lecithin is 1:2.

9. The controlled dosage aerosol according to claim 1 for treating allergic diseases in humans and animals.

10. The controlled dosage aerosol according to claim 1 for treating asthma or allergic rhinitis.

11. A process for the production of controlled dosage aerosols according to claim 1, comprising the steps of:
mixing isobutane as a propellant in liquid form, lecithin as a surface-active agent in liquid form, and at least one medicinal agent as solid substance to form a liquid suspension; and
filling said liquid suspension under pressure into a spray tin having a valve.

12. The process according to claim 11, wherein the temperature of said liquid suspension after said filling step is between −10 and +10° C.

13. The process according to claim 11, further comprising the step of cleaning said valve of said spray tin by filling said spray tin up with a propellant, said cleaning step being after said filling step.

14. The controlled dosage aerosol according to claim 8, wherein the ratio of glucocorticoid and soybean lecithin is 1:1.

15. The controlled dosage aerosol according to claim 14, wherein the ratio of glucocorticoid and soybean lecithin is 1:0.5.

16. The controlled dosage aerosol according to claim 9 for inhalation treatment of allergic diseases of the respiratory tract.

17. A process for treating allergic diseases comprising administering a controlled dosage aerosol, said aerosol including at least one medicinal agent, a propellant, and lecithin, said lecithin being a surface-active agent, and wherein said at least one medicinal agent is in the form of a suspension and said propellant is pressure-liquefied isobutane.

18. The process for treating allergic diseases according to claim 17, wherein said allergic diseases are selected from the group consisting of allergic rhinitis and asthma.
